(19) United States
(12) Patent Application Publication
(10) Pub. No.: US 2006/0173331 A1
(43) Pub. Date: Aug. 3, 2006

(54) Ergonomic transducer housing and methods for ultrasound imaging

(75) Inventors: Brian E. Booton, San Jose, CA (US); Joseph J. Molinari, Redwood City, CA (US); Gail Kathleen Quiroz, Redwood City, CA (US); Dean J. Bidwell, Renton, WA (US); Andrew L. Walston, Seattle, WA (US); Kathryn M. Kuebler, Seattle, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/286,538

(22) Filed: Nov. 23, 2005

Related U.S. Application Data
(60) Provisional application No. 60/630,729, filed on Nov. 24, 2004.

(51) Int. Cl. A61B 8/14

(52) U.S. Cl. 600/445

(57) ABSTRACT

A palmar surface may allow for ease of gripping an ultrasound transducer probe. By facing the palmar surface so that the users hand is directed downwards, towards the patient's skin, during use, less wrist flexing may result. To allow for gripping, the cable extends from the transducer at a location other than the top of the transducer. Elastomer or other soft materials increase grip.

### RELATED APPLICATIONS

The present patent document claims the benefit of the filing date under 35 U.S.C. § 119(e) of Provisional U.S. Patent Application Ser. No. 60/630,729, filed Nov. 24, 2004, which is hereby incorporated by reference.

### BACKGROUND

This present embodiments relate to ultrasound transducers. In particular, ultrasound imaging transducer housings or form factors have an ergonomic shape.

Handheld ultrasound transducers for medical imaging include an array of transducer elements and associated electronics. The transducer housing generally conforms to the enclosed array and electronics. A broader acoustic window region houses the array and electronics. A narrower, cylindrical region extends away from the acoustic window for gripping by the user. The sonographers use pinch grips and wrist flexing to obtain the desired ultrasound images.

Different sized grip regions may be provided. The different sizes may increase comfort for the users. However, the different sizes still require uncomfortable pinch grips and excessive wrist flexing as the sonographer positions the transducer relative to the patient to scan the desired tissue.

### BRIEF SUMMARY

By way of introduction, the preferred embodiments described below include a methods, transducers and transducer housings for ergonomic ultrasound imaging. A palmar surface may allow for ease of gripping the transducer. By facing the palmar surface so that the users hand is directed downwards, towards the patient's skin, during use, less wrist flexing may result. To allow for gripping, the cable extends from the transducer at a location other than the top of the transducer.

In a first aspect, a method is provided for ultrasound scanning with a transducer. An upper surface of the transducer is positioned in a palm of a user with the upper surface shaped to allow at least the thumb, a finger or thumb and finger of the user to extend off the upper surface in a direction generally down towards an acoustic window of the transducer. The acoustic window of the transducer is a lower surface and the upper surface being substantially opposite the lower surface. The transducer moves in response, at least in part, to force applied by the palm of the user against the upper surface.

In a second aspect, a transducer housing is provided for ultrasound imaging. An acoustic window allows for positioning an array of transducing elements adjacent to skin of a patient. A palmar surface is substantially parallel with the acoustic window.

In a third aspect, a transducer housing is provided for ultrasound imaging. An acoustic window allows for positioning an array of transducing elements adjacent to skin of a patient. A palmar surface has a maximum circumference in a first plane substantially parallel with the acoustic window. A cross sectional area substantially aligned with the maximum circumference is greater than any other cross sectional area in planes substantially parallel but different from the first plane.

In a fourth aspect, a transducer housing is provided for ultrasound imaging. An acoustic window allows for positioning an array of transducing elements adjacent to skin of a patient. An orthogonal vector extends from a center of the acoustic window. A palmar surface intersects the orthogonal vector. A cable extends from the housing at a location spaced away from the orthogonal vector.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIGS. 1A and 1B illustrate side and perspective views of one embodiment of a palmer grip on a transducer;

FIG. 2 shows side, perspective and use views of another embodiment of transducer with a palmer grip;

FIG. 3 shows side, top, perspective and use views of an embodiment of a transducer having a palmer ball form factor;

FIG. 4 illustrates a perspective and a use view of one embodiment of a transducer having an incorporated palmer resembling a gear shifter housing;

FIG. 5 shows top and side views of a palmer grip of another embodiment with an offset strain relief curling over a user's hand;

FIG. 6 shows top, bottom and side views of another embodiment of a palmer grip with a spherical design and an offset cable exit;

FIG. 7 shows side views of yet another palmer grip embodiment with modified spherical form factor;

FIG. 8 illustrates perspective and use views of another embodiment of a palmar surface;

FIG. 9 shows side views of a palmer grip half sphere embodiment;

FIG. 10 shows side views of a hybrid palmer grip spherical end and traditional handle embodiment;

FIG. 11 shows side views of a palmer grip with offset cable exit embodiment;

FIG. 12 shows side views of an alternative hybrid palmer grip with a large spherical end;

FIG. 13 shows side views of one embodiment of a palmer or ball grip transducer with angled and rotatable strain relief incorporated into elastomer of a rear handle;

FIG. 14 shows side views of another embodiment of a palmer or ball grip transducer with 90 degree angled and rotatable strain relief incorporated into elastomer of a rear handle;

FIG. 15 shows side views with partial cut-aways and a perspective view of a modular elastomer handle that has inverse strain relief to limit cable bend radius, which centralizes mass and reduce cable weight torque on wrist;

FIG. 16 shows side and top views of one embodiment of a linear transducer with rotating rear housing and angled strain relief;

FIG. 17 shows side views of another embodiment of a palmer grip transducer with angled strain relief and rotating rear housing;

FIG. 18 illustrates side, perspective and use views showing an embodiment transducer having a handle that pivots for less torsional strain when the transducer is micro-positioned;

FIG. 19 is an exploded view of one embodiment of the transducers of FIG. 16 or 17;

FIG. 20 shows side views of one embodiment of a transducer with separate elastomer grip that can be repositioned at different angles;

FIG. 21 shows one embodiment of a modular transducer housing;

FIG. 22 illustrates a use view of one embodiment of a transducer housing to provide strain relief between the user's fingers;

FIG. 23 shows side views of an embodiment of a transducer incorporated with a combined palmer form factor and elastomer elements;

FIG. 24 illustrates gripping elements on an ultrasound transducer in accordance with another embodiment;

FIG. 25 illustrates incorporation of elastomers materials on an ultrasound transducer in an alternate embodiment;

FIG. 26 illustrates incorporation of cushioning materials on the body of an ultrasound transducer in accordance with one embodiment;

FIG. 27 shows different embodiments of a transducer gripper elements; and

FIGS. 28-29 illustrate embodiments of endocavity transducers having ergonomic elements.

### DETAILED DESCRIPTION OF THE DRAWINGS AND PRESENTLY PREFERRED EMBODIMENTS

Ergonomic transducer housings may prevent injury and/or increase comfort for sonographers. A wide range of ultrasound applications, including 2-D, 3-D and 4-D scanning applications, use handheld transducer housings. During an ultrasound study, the sonographer makes a series of sweeping motions, macro and micromanipulations, superior to inferior angulations and lateral to medial rotations. The sonographer may use gloves and ultrasound gel, making gripping of the transducer housing during the motions difficult. Palmer grips may allow more secure gripping. Palmar grips with or without soft touch elastomer materials and/or removable modular rear handle attachments may provide ergonomic industrial design transducer solutions. A palmar surface on the transducer housing allows gripping the transducers over a portion or the entire rear case form factor of the transducer. The transducer housings may facilitate a wide range of hand sizes with removable modular rear handle attachments.

Sonographers often depend on repetition and develop scanning stances that vary little from patient to patient. Within each exam, however, there may be a range of hand positions appropriate to the particular application. In some applications, a particular embodiment of a transducer may be more desirable. For example, with ultrasound imaging applications such as obstetrics/gynecology and abdomen applications, embodiments of transducers have the transducer rear case optimized for comfort and usability, such as including a palmer grip form factor. The ergonomic shape of the palmer grip provides greater comfort to the user when pressure is applied to the transducer. An elastomer material may allow for better comfort and easier gripping of the transducer.

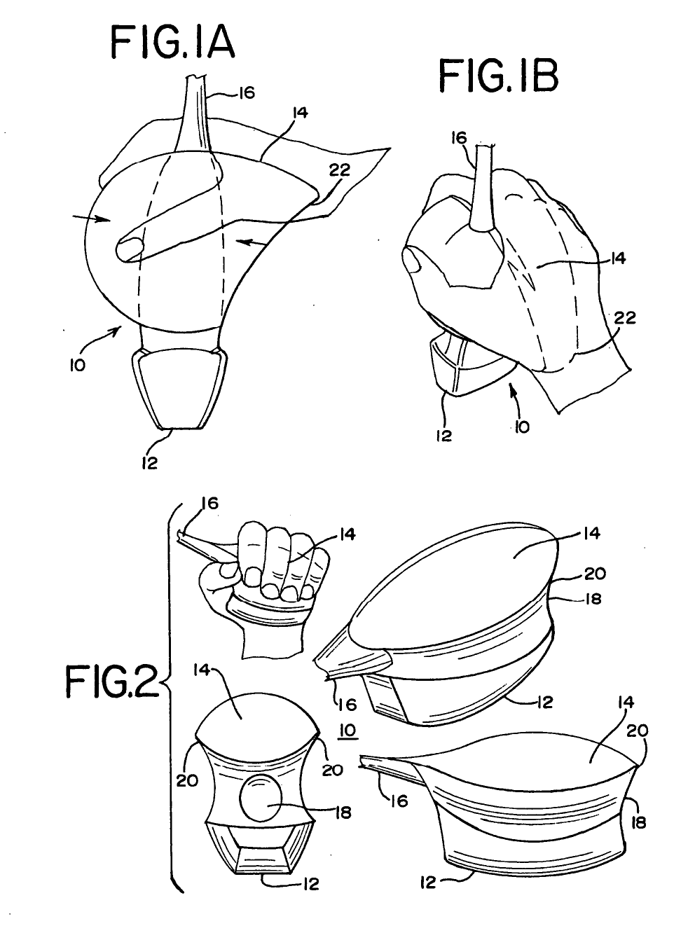

FIGS. 1A, 1B and 2 show a transducer housing 10 for ultrasound imaging. The transducer housing 10 is for handheld use external to the patient. The sonographer grips or holds the transducer housing 10 for scanning a patient along the surface of the skin. In alternative embodiments, the transducer housing 10 is a part of an endocavity, intraoperative or other transducer housing with a portion of the housing used internally of the patient. The ergonomic aspects of the transducer housing are shaped for user gripping within the patient (e.g., intraoperative probe) or for holding a portion of the probe that is external to the patient (e.g., transesophageal probe).

The transducer housing 10 includes an acoustic window 12, a palmar surface 14, a cable 16, and a finger grip 18. Additional, different or fewer components may be provided. The transducer housing 10 is plastic, metal, wood, fiberglass, epoxy, resin, combinations thereof or other now known or later developed material. Single piece or multiple pieces form the transducer housing 10. For multiple pieces, snap fit, screws, glue, epoxy, nuts and bolts, or other now known or later developed connections between different pieces are provided. For example, the acoustic window 12 snap fits with the rest of or is formed integral with the transducer housing 10.

The acoustic window 12 is plastic, epoxy, resin, glass or other material acoustically matched to a transducer array and/or the patient. The acoustic window 12 in alternative embodiments is an aperture or opening for allowing the transducer array access to the patient for scanning. The acoustic window 12 is positioned adjacent to tissue, such as the skin, of a patient during use. The transducer array within the transducer housing 10 is adjacent to the acoustic window 12. The positioning of the acoustic window 12 positions the array for scanning the patient.

The acoustic window 12 is flat, planar, curved along only one dimension or curved along two dimensions. An orthogonal vector conceptually extends from a center of the acoustic window 12. In FIG. 1, the orthogonal vector extends from the acoustic window 12 to the cable 16. The orthogonal vector is generally a central axis of the transducer housing 10.

The palmar surface 14 is shaped to fit a palm of a user. For example, the palmar surface is rounded or elliptical generally in conformance with a palm shape. A continuous gradual curve as shown in FIGS. 1A, 1B and 2 may be used. Alternatively, a more complex surface more closely emulating the palm shape may be used. The palmar surface 14 is smooth, but ridges, crevices, divots, bumps or other structures may be provided.

The palmar surface 14 is sized as appropriate for a user's hand. For example, the palmar surface 14 extends along a longitudinal dimension from a base of a palm to where the user's fingers connect with the palm, to the first joint of the users fingers or another length. As another example, the palmar surface 14 has a width a little less than, at or a little wider than the palm, allowing the thumb to partially or completely extend off the palmar surface 14.

In one embodiment shown in FIGS. 1A and 1B, the palmar surface 14 extends gradually around the transducer housing 10 for placement of the palm, fingers and thumb. Friction or pressure is used to hold the transducer housing 10 with the palmar surface 14 adjacent a user's palm. In another embodiment shown in FIG. 2, a lip 20 extends along an edge of the palmar surface 14. The lip 20 corresponds generally to a change in surfaces, such as a ledge or alteration in direction or curvature by over 45, 90, 120 or other number of degrees. The change is immediate, such as a corner, or is gradual, such as being rounded. The lip 20, at least in part, is for gripping the transducer housing around the palmar surface 14. The lip 20 allows the fingers and/or thumb to apply a force upwards or away from the acoustic window 12 while the user's palm is positioned on the palmar surface 14.

For use of the transducer housing 10, the palmar surface 14 intersects the orthogonal vector from the acoustic window 12. A least a portion of the user's hand is positioned on an upper or top surface or point of the transducer housing 10. For example and as shown in FIGS. 1A, 1B and 2, the palmar surface 14 is substantially parallel with the acoustic window 12. The directions of curvature may be different or opposite between the acoustic window 12 and the palmar surface 14, but both are generally parallel (e.g., on the bottom and on the top of the transducer housing 10). Being on a top of the transducer housing 10, the palmar surface 14 is generally parallel with the skin of the patient for use of the transducing elements. The user orients their palm generally facing towards skin of a patient and over the palmar surface 14. The palmar surface 14 may be symmetric about or centered on the orthogonal vector. Alternatively, the palmar surface 14 is offset, such as having an edge region adjacent the intersection of the orthogonal vector with the top of the transducer housing and an opposite edge spaced further away from the intersection. In yet another alternative embodiment, the palmar surface 14 does not intersect the orthogonal vector.

Since the transducer housing 10 is to be held with the user's palm against or adjacent to the palmar surface 14, the palmar surface 14 is generally a largest contiguous surface of the transducer housing 10. For example and as shown in FIG. 2, a cross section through the palmar surface 14 provides a maximum circumference of the transducer housing 10 wherein the cross section is in a plane substantially parallel with the acoustic window 12. The cross sectional area through the palmar surface 14 is greater than any other cross sectional area in planes substantially parallel but different than the first plane. As another example and as shown in FIGS. 1A and 1B, a surface area of the palmar surface 14 is greater than any other contiguous surface. Ridges, lips or other distinctions separate the different connected surfaces.

The finger grip 18 is a shaped area, such as an indentation, ridges, bumps, dimples, crevices or other structure for accepting the fingers and/or thumb of the user. In one embodiment, the finger grip 18 is formed of the same material as the transducer housing 10. In other embodiments, other materials form the finger grip, such as an elastomer pad bound to the transducer housing. The elastomer pad may extend beyond the finger grip 18, such as covering all or part of the palmar surface 14.

The finger grip 18 is between the acoustic window 12 and the palmar surface 14. When the user positions their hand over the palmar surface, the fingers or thumb extend from the palmar surface 14 to the finger grip 18. A single or multiple finger grips may be provided. The lip 20 may act as a finger grip 18. In alternative embodiments, no finger grip 18 other than an overall shape of the transducer housing 10 is provided.

The palmar surface 14 with or without the lip 20 and finger grip 18 form an ergonomic shape of the transducer housing 10. A larger area of contact has no spots of local high pressure (i.e., no substantial ridges not associated with the natural curvature of the hand). The user's grip fits naturally over the transducer housing 10, minimize narrow pinch grips (e.g., pencil or joystick type grips). A power grip or modified power grip may be used. Hilts, pommels, or central thickening features allow momentary relaxation of grip. Multiple handgrips may be used on transducer power or precision style grips. The transducer housing 10 may be gripped securely without having to rely on friction.

The cable 16 is a bundle of coaxial cables, a ribbon cable, twisted wire cable, single coaxial cable, combinations thereof or other now known or later developed conductors. The cable 16 includes a flexible cover, such as rubber, plastic or elastomer cover. The cable 16 may include a strain relief at a connection with the transducer housing 10. The cable 16, such as the strain relief portion of the cable, has a circular, elliptical, tapered or oblong shaped. For example, the cable 16 has an elliptical or oblong shape adjacent to the transducer housing 10 to fit between the user's fingers or finger and thumb.

The cable 16 extends from the transducer housing 10 along the orthogonal vector (i.e., at a center location on the top of the transducer housing 10) or at an offset location. An offset location is spaced away from the orthogonal vector. For example, the cable 16 extends from the transducer housing at an angle substantially perpendicular to the orthogonal vector as shown in FIG. 2. The location of the intersection of the cable 16 with the transducer housing 10 is on a side or top of the transducer housing 10.

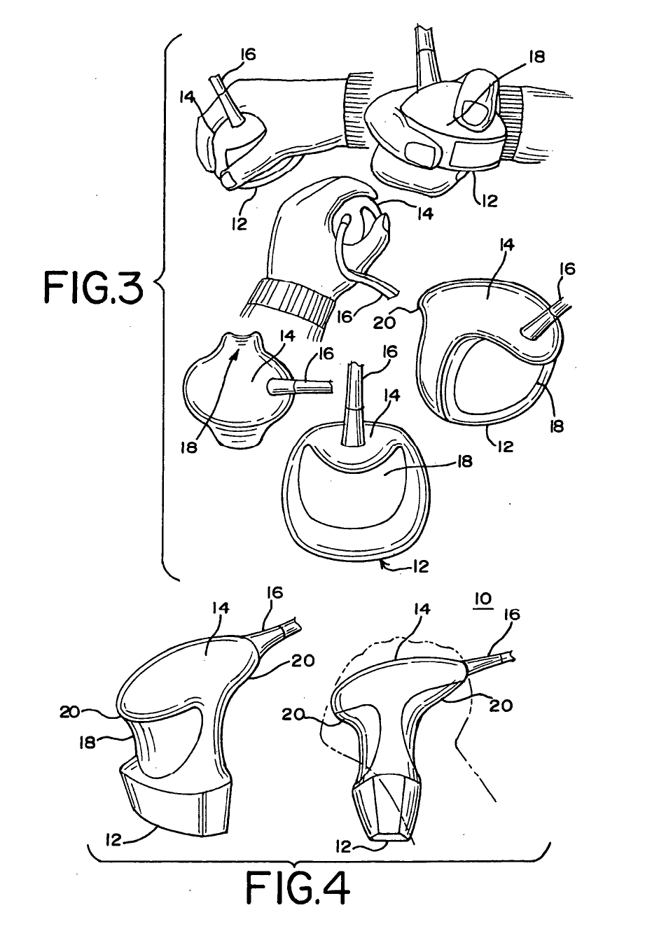

The location is on or off the palmar surface 14. For example, the location is between the palmar surface 14 and the acoustic window 12 as shown in FIG. 2. As another example, the location is centered on the palmar surface 14 as shown in FIGS. 1A and 1B. As yet another example, the location is on an edge or off-center location of the palmar surface as shown in FIG. 3. The position of the location and angle of the cable 16 may reduce cable strain on musculoskeletal structure of user. The relative location of the palmar surface 14 concentrates the mass and forces, including cable strain forces, of the transducer probe inside the grip area.

The palmar surface 14 of FIGS. 1A and 1B includes a ledge 22 feature under the user's palm to allow the user to exert more force on the probe with advantage comfortably during use. The large ball structure of the palmar surface 14 reduces the amount of pinching for controlling the transducer housing 10. The palmar surface 14 of FIG. 2 uses a smaller sizing, allowing the user to wrap more fully their hand around the transducer housing 10 or palmar surface 14. The grasp with the palmar surface 14 avoids pinching the transducer housing 10 between the fingers and thumb.

FIGS. 1A, 1B and 2 show two different embodiments of transducer housings 10 with palmar surfaces 14. Many other embodiments are possible.

FIG. 3 shows different views of another embodiment of the transducer housing 10. The transducer housing 10 and palmar surface generally have a palmer ball form factor. The finger grip 18 merges with the palmar surface 14 for orientation of the transducer housing 10 relative to the user. Alternatively, the merged finger grip 18 is not provided. A textured elastomer extends or is molded over all or most of the transducer housing 10 or palmar surface 14. The side finger grips 18 also or alternatively included textured elastomer material. The pinch grip is reduced or eliminated by providing a form factor held more like a tennis ball. For one embodiment, the palmer ball form factor has a low profile, which allows for a lower center of gravity for the probe. As a result, strain associated with micro positioning the probe may be decreased.

FIG. 4 shows one embodiment of the transducer housing 10 having a gear shifter shape, such as a car gear shifter. The finger grip 18 is sized and shaped for a thumb generally parallel to the acoustic window 12. The fingers extend to the same finger grip 18 and/or a lip 20 over the palmar surface 14. The cable 16 extends from the transducer housing 10 along an axis surrounded by the users hand during use.

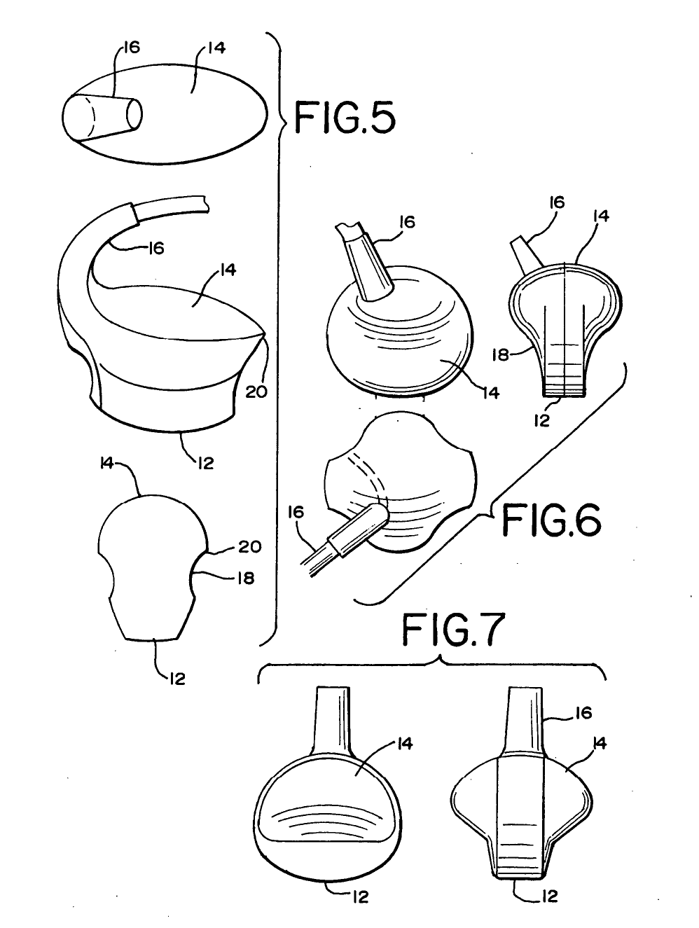

FIG. 5 shows another embodiment of the transducer housing 10. The cable 16 extends from an edge of the palmar surface 14, such as at a tip of an elliptical shaped palmar surface 14 for positioning between the fingers and thumb. The fingers wrap around the transducer housing 10 with the palm and palmar surface 14 on a top of the transducer housing 10, normalizing the user's wrist angle during use. The size is generally small, such as an elongated tennis ball or about 2-3 inches in height.

FIG. 6 shows another embodiment of the transducer housing 10. The cable 16 extends from the palmar surface 14 on an edge area. The palmar surface 14 distributes load while allowing fingertip control. The acoustic window 12 extends from the ball shaped upper portion. FIG. 7 shows another embodiment similar to FIG. 6, but with the cable 16 positioned in the center of the palmar surface 14. The palmar surface 14 has a 2.8 inch or other radius perpendicular to the acoustic window 12, but a 40 mm or other diameter parallel with the acoustic window 12. FIG. 8 shows another similar embodiment with the cable 16 at an off-center location on the palmar surface 14. A dip or indention in the palmar surface 14 at the cable 16 may allow the cable to flex or bend out the way of the user's hand when gripping the transducer housing 10.

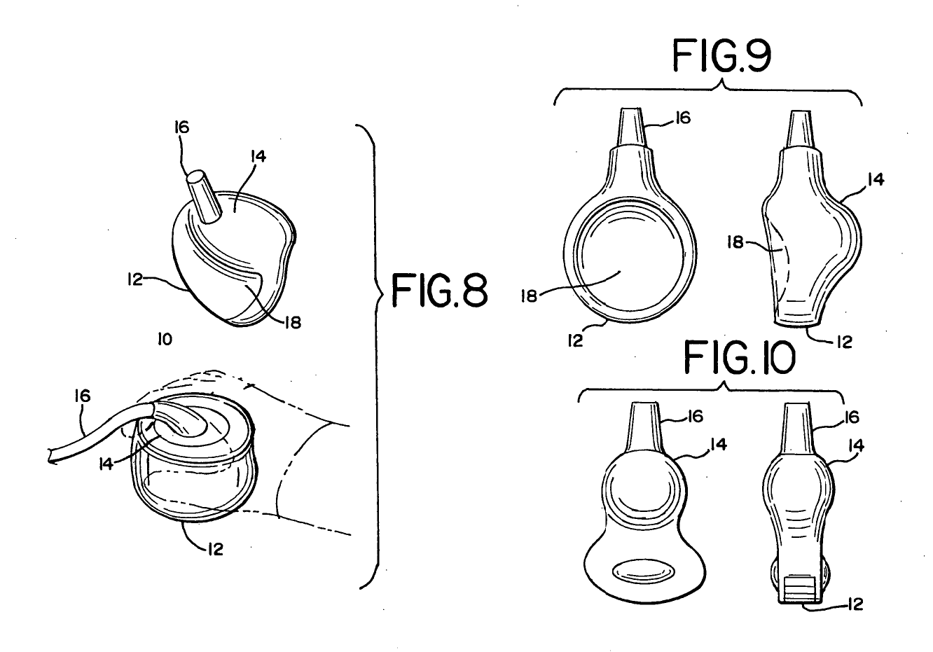

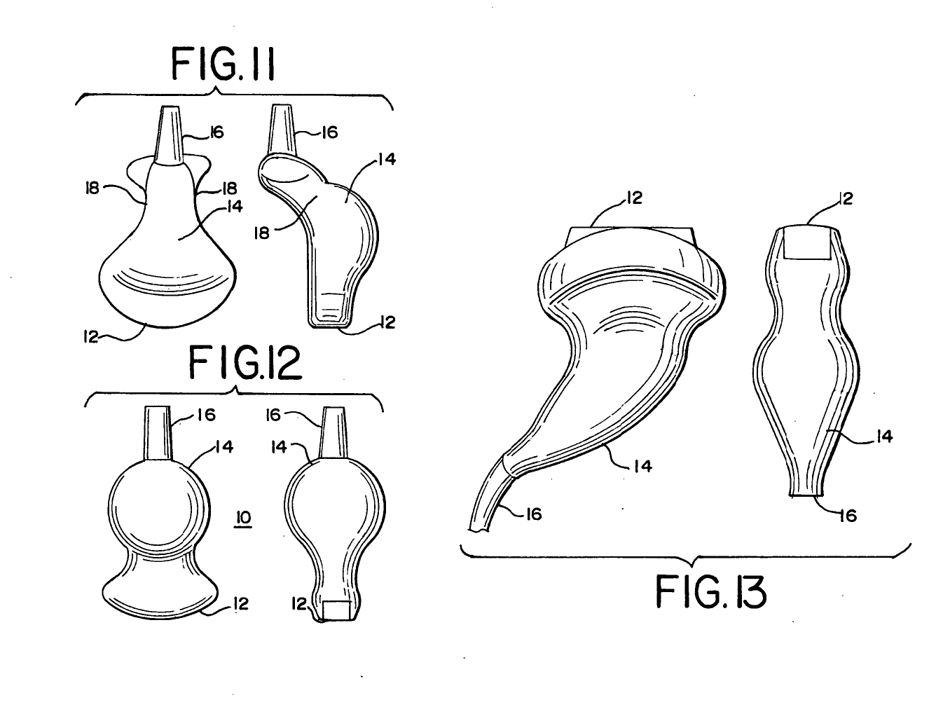

FIG. 9 shows an embodiment of the transducer housing 10 where the palmar surface 14 is tilted or on a side of the transducer housing 10. A thumb indention is a finger grip 18 for positioning the user's hand with the centered transducer cable between the fingers and thumb. FIG. 10 shows an alternative with a general small ball shape for a centered palmar surface 14 and centered cable 16. The user grasps the palmar surface 14 rather than using a pinch grip. FIG. 11 shows another embodiment with the palmar surface 14 on a side of the transducer housing 10 with two finger grips 18 or indentations along the sides of a more upper portion. The cable 16 extends from an offset location at the top of the transducer housing 10 from between the finger grips 18.

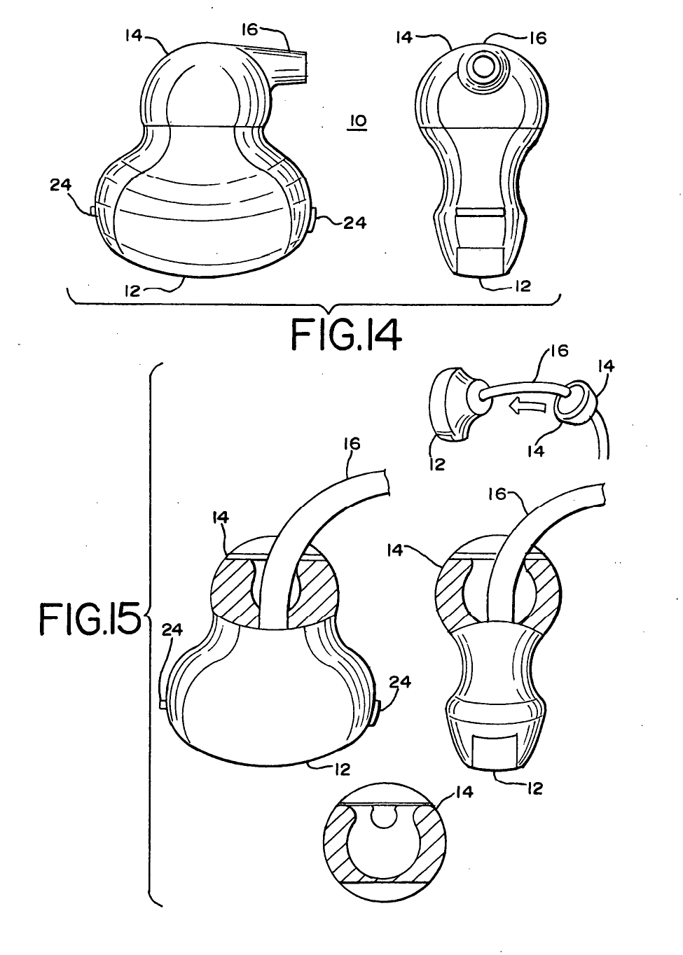

FIG. 12 shows an embodiment similar to FIG. 10, but with a larger palmar surface 14. The larger palmar surface 14 either allows for or does not allow for the fingers and/or thumb to extend off the palmar surface 14 or ball region of the transducer housing.

FIG. 13 shows another embodiment of the transducer housing 10. The strain relief of the cable 16 smoothly and gradually merges into or with the palmar surface 14. The cable 16 extends at any desired direction from the transducer housing 10 relative to the acoustic window 12. For example, the cable 16 extends along a dimension along which the array of elements and acoustic window 12 extend. The strain relief and/or a portion of the palmar surface 14 are flexible.

FIG. 14 shows another embodiment of the palmar surface 14 having a generally ball shape. The cable 16 extends from the palmar surface 14 at a perpendicular angle to the orthogonal or center axis of the transducer housing 10. The transducer housing 10 may have different colors, such as to identify the intended grip area or palmar surface 14. Biopsy features 24, such as depressions to hold a biopsy guide, are positioned on the sides of the transducer housing 10, such as adjacent to the acoustic window 12.

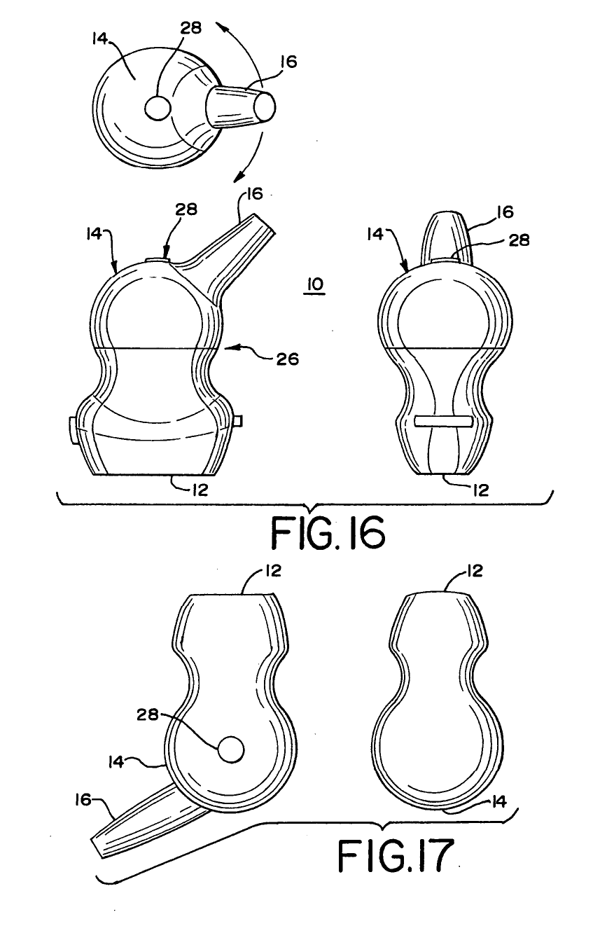

FIG. 15 shows an embodiment with the palmar surface 14 being detachable from the rest of the transducer housing 10 for cleaning. Threading, snap fit or other releasable attachment is provided. The palmar surface 14 has a 46 mm diameter, but other sizes may be used. A micro strain relief of the cable 16 allows flexing of the cable 16 for more comfortable gripping of the palmar surface 14. The strain relief is within an indentation in the palmar surface 14, such as being on the portion of the transducer housing 10 from which the palmar surface is removed. A cable snap or indentation on the palmar surface holds the cable in one specific direction. The cable 16 snaps into the indentation.

FIG. 16 shows an alternative embodiment of the transducer housing 10. The palmar surface 14 is rotatable along a joint 26. The palmar surface 14 is covered by or made from elastomer. The cable 16 extends from an off-center location. The cable 16 and palmar surface 14 rotate relative to the acoustic window 12 for comfort or use. The acoustic window 12 is flat, such as associated with a 40 mm or other size linear array. A color marker 28 indicates the type of transducer. Alternatively, a label is provided.

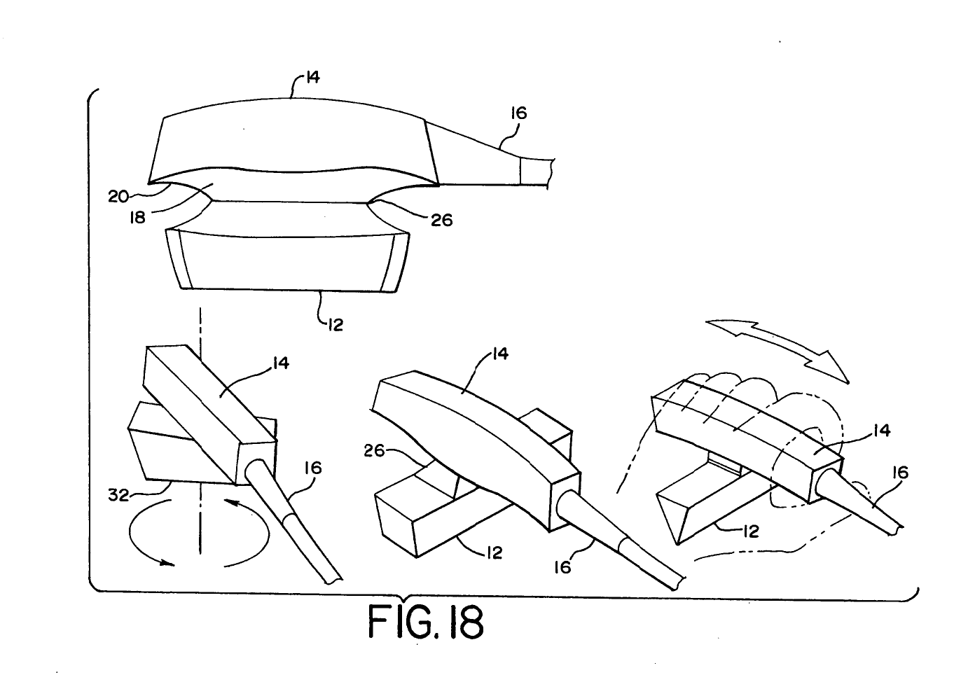

FIG. 17 shows another embodiment of the transducer housing 10 with a color marker 28. The color marker 28 is positioned on the side of a ball shaped palmar surface 14.

FIG. 18 shows an embodiment of the transducer housing 10 with a rotatable joint 26. The palmar surface 14 rotates relative to the acoustic window 12 at the joint 26. The rotation has sufficient friction, indentations or other settings for holding the relative position during use. The finger grip 18 is provided beneath the lip 20 above, below or both above and below the joint 26. During use, the palmar surface 14 may be at a generally right (i.e. 90 degree) angle to the acoustic window 12 and associated array, making rocking movement along the axis of the array convenient for small adjustments in scan plane without translation. The cable 16 extends from the palmar surface along an axis of the hand during use, creating a lower center of gravity for less torsional strain during any rocking motion.

FIG. 19 shows one arrangement of transducer probe components for the rotatable joint 26. The color mark or other cap 26 is positioned to snap or be bonded over a fastener 38, such as a screw or bolt. The fastener 38 extends through a cap 40. The cap 40 provides the palmar surface 14 and the strain relief of the cable 16. The wires, coaxial cables or other conductors 42 from the cable 16 are looped in a full circle. Greater or less looping may be provided. A fastener or hold-down 44 maintains a position of the terminations relative to the acoustic window 12 and associated array. As the cap 40 is rotated, the loop of the conductors 42 changes diameter about the fastener 38. A double seal 46 seals the cap 40 to the seal ledge 52. A single or other seals may be used. A pivot fastener 48 fixedly connects with the transducer housing 10 of the acoustic window 12 between the seals 46. The fastener 38 fastens into an upwardly extending portion of the pivot fastener 48. The cap 40 rotates about the pivot fastener 48. A rotation limiter 50 prevents over winding of the loop of conductors 42 by limiting the rotation of the cap 40 to less than 361 degrees. Other now known or later developed arrangements or components for rotation may be used.

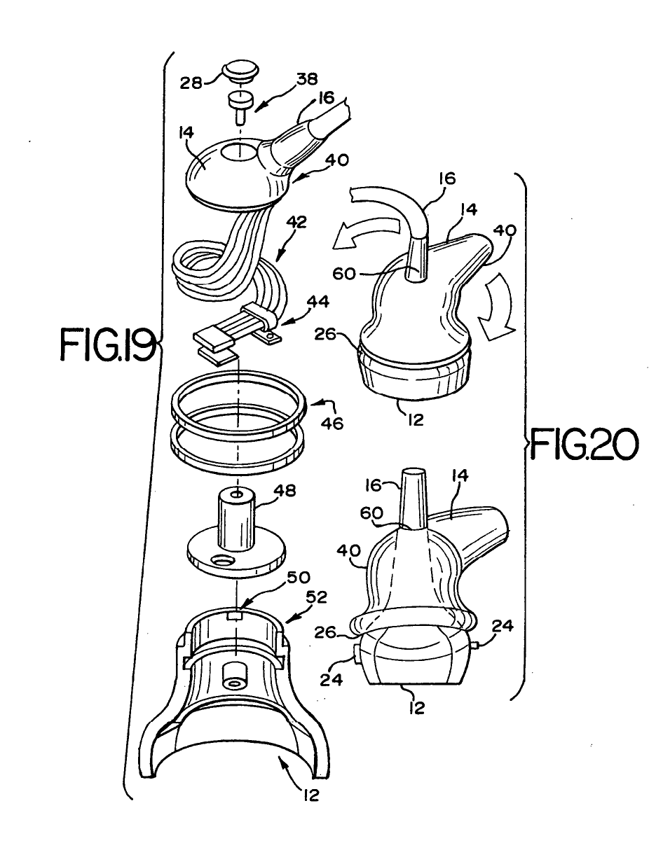

FIG. 20 shows an embodiment of the transducer housing 10 with a cable connector 60 connecting the cable 16 to the transducer housing 10. The cable connector 60 rotates such that the cable 16 may rotate relative to the transducer housing 10. Alternatively, the cable 16 connects fixedly to the transducer housing 10. A rotatable joint 26 also allows the palmar surface 14 to swivel relative to the acoustic window 12. A cylindrical internal arrangement allows ease of rotation while a lever arm 22 of the palmar surface 14 provides comfortable control of the rotation and gripping. The cap 40 is removable, such as by un-fastening a fastener.

FIG. 21 shows the cap 40 with the palmar surface 14 for removal. Different sized and/or shaped caps 40 are provided. An internal handle, such as a cubic handle, contains the acoustic window 12, associated electronics and cable 16. The cap 40 snap fits or slides onto the internal handle to provide the desired palmar surface 14. For example, any of the embodiments shown in U.S. Pat. No. 6,237,192, the disclosure of which is incorporated herein by reference, may be used.

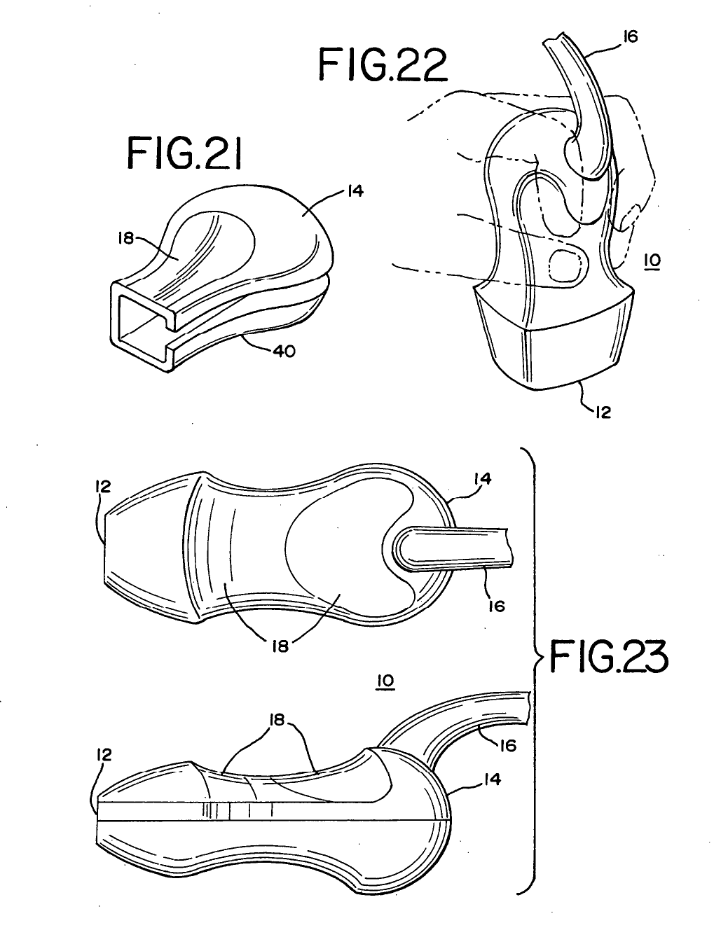

FIG. 22 shows the transducer housing 10 with a cable 16 offset from a center-top location. The cable 16 extends upward, such as parallel to an orthogonal from the acoustic window 12, but spaced away from a center location. For positioning between the fingers or fingers and thumb, the strain relief of the cable 16 has an oval or elliptical shape with a major axis being perpendicular to but intersecting the center or orthogonal. Other alignments may be used.

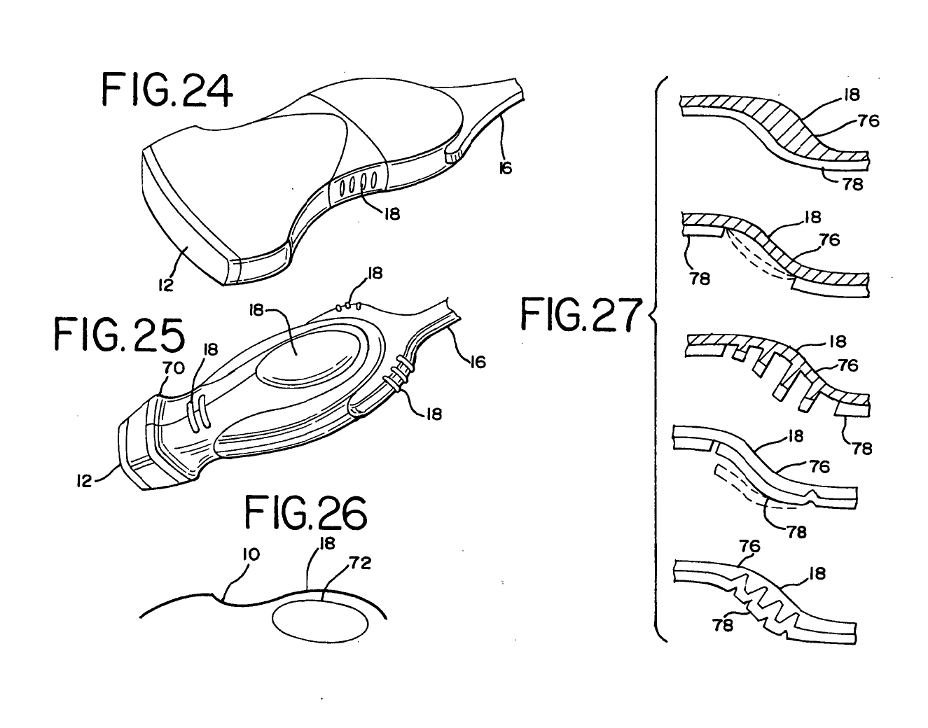

FIG. 23 shows the transducer housing 10 with elastomer finger grips 18. The finger grips 18 have a roughened or textured surface. The ergonomic palmer grip 14 and elastomeric components (e.g., finger grips 18) may provide an improved ergonomic ultrasound transducer form factor. FIG. 24 shows finger grips 18 with elastomer ridges positioned towards a top of the transducer housing 10. This positioning may allow for different or more comfortable gripping, such as where a palmar surface 14 is on the top of the transducer housing 10. The positioning may also be used as shown without a palmar surface 14. FIG. 25 shows other positions for elastomer ridges of finger grips 18. Ridges on the strain relief of the cable 16 allow for more downward gripping. The elastomer ridges of the lower finger grips 18 are backlit for easy visibility in dark exam rooms. Frosted, colored or clear elastomer is used for backlighting. Elastomer creates a rounded edge 70 to soften the edge 70.

FIG. 26 shows a gel-filled pocket 72 positioned under elastomer or other flexible material in the center finger grip 18 of FIG. 25. The gel-filled pocket 72 allows the finger grip 18 to conform more comfortably to the user's hand. FIG. 27 shows alternative embodiments for conforming the finger grip 18 to the user's hand for comfort. Different plastic or other material sub frames 78 support elastomer (e.g., TPE) or other soft material 76. A thickened region of elastomer 76 overmold, a hole in the sub frame 78 allowing greater flexing of the elastomer 76, ribbing of the sub frame 78 and different thickness of the elastomer 76, a flexible or hinged sub frame 78, or textured sub frame 78 assist gripping and provide user comfort. Ridges, gel filled pockets or other gripping structures or texture may be provided in other locations, such as over the entire palmar surface 14 or the entire housing 10. Material other than or in addition to elastomer may be used.

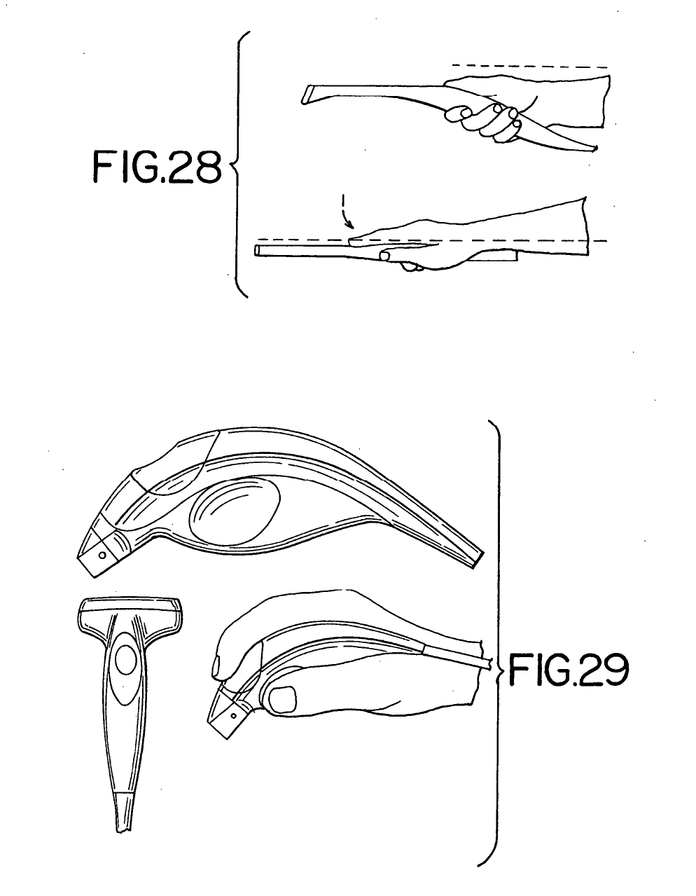

FIGS. 28 and 29 show embodiments of endocavity transducers having ergonomic elements. The transducer of FIG. 28 is handled like a wand or pointer. For one grip, extending the pointer finger out along the axis of the transducer makes the center of rotation more intuitive. The embodiment of FIG. 29 may be used for a neonatal probe for increased clearance in isolet.

For some embodiments with a palmar surface, a method is provided for ultrasound scanning with a transducer. An upper surface of the transducer is positioned in a palm of a user. The upper surface allows at least the thumb, a finger or thumb and finger of the user to extend off the upper surface in a direction generally down towards an acoustic window of the transducer. The acoustic window of the transducer is a lower surface with the upper surface being substantially opposite the lower surface. The upper surface is rounded generally in conformance with a palm shape. Other positioning of the user's hand with or without a palmar surface may be used.

An elastomer pad is beneath or lower than the upper surface on a side surface. The user positions a thumb or finger adjacent to the elastomer pad. For example, elastomer material is at one or more locations on the upper or palmar surface.

After ergonomically gripping the transducer housing, the transducer moves in response, at least in part, to force applied by the palm of the user against the upper surface. For example, the user pushes against the upper surface. As another example, the user orients the transducer with the palm of the user generally facing towards skin of a patient. Movement occurs while the user grips the transducer around the upper surface of the transducer.

The cable extends from the transducer at a location spaced away from an orthogonal to a center of the acoustic window to allow gripping around the upper surface. Alternatively, the palmar surface is tilted or oriented such that the cable may extend from a center location without interfering with the palm, fingers or thumb of the user.

The transducer housings are used during ultrasound study with a series of broad sweeping motions, macro and micromanipulations, superior to inferior angulations and/or lateral-to-medial rotations. The user may use the transducer housings with gloves and ultrasound gel. The comprehensive ergonomic transducer housings provide physical form factors and soft touch elastomer materials to address this environmental state. Alternatives to the traditional pinch gripping and wrist flexing are provided.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for ultrasound scanning with a transducer, the method comprising:
positioning an upper surface of the transducer in a palm of a user with the upper surface shaped to allow at least the thumb, a finger or thumb and finger of the user to extend off of the upper surface in a direction generally down towards an acoustic window of the transducer, the acoustic window of the transducer being a lower surface and the upper surface being substantially opposite the lower surface; and
moving the transducer in response, at least in part, to force applied by the palm of the user against the upper surface.

2. The method of claim 1 further comprising:
positioning an elastomer pad beneath the upper surface on a side surface in a position adjacent the thumb or finger.

3. The method of claim 1 wherein moving comprises pushing against the upper surface.

4. The method of claim 1 wherein moving comprises orienting the transducer with the palm of the user generally facing towards skin of a patient.

5. The method of claim 1 wherein moving comprises gripping the transducer around the upper surface of the transducer.

6. The method of claim 1 further comprising:
attaching a cable extending from the transducer at a location spaced away from an orthogonal to a center of the acoustic window.

7. The method of claim 1 further comprising:
positioning elastomer material at one or more locations on the upper surface.

8. The method of claim 1 wherein positioning the upper surface comprises positioning the upper surface with the upper surface being rounded generally in conformance with a palm shape.

9. A transducer housing for ultrasound imaging, the transducer housing comprising:
an acoustic window for positioning an array of transducing elements adjacent to skin of a patient;
a palmar surface substantially parallel with the acoustic window.

10. The transducer housing of claim 9 wherein the palmar surface is generally parallel with the skin for use of the transducing elements.

11. The transducer housing of claim 9 further comprising at least one finger grip between the acoustic window and the palmar surface.

12. The transducer housing of claim 9 further comprising an elastomer pad on the palmar surface, between the acoustic window and the palmar surface or both.

13. The transducer housing of claim 9 wherein the palmar surface is shaped to fit a palm of a user oriented with the palm of the user generally facing towards skin of a patient.

14. The transducer housing of claim 9 further comprising a lip along an edge of the palmar surface, the lip for gripping the transducer housing around the palmar surface.

15. The transducer housing of claim 9 further comprising:
a cable extending from the transducer housing at a location on or off of the palmar surface, the location spaced away from an orthogonal to a center of the acoustic window.

16. The transducer housing of claim 9 wherein the palmar surface is rounded generally in conformance with a palm shape.

17. A transducer housing for ultrasound imaging, the transducer housing comprising:
an acoustic window for positioning an array of transducing elements adjacent to skin of a patient;
a palmar surface having a maximum circumference in a first plane substantially parallel with the acoustic window, a cross sectional area being substantially aligned with the maximum circumference greater than any other cross sectional area in planes substantially parallel but different than the first plane.

18. The transducer housing of claim 17 wherein a surface area of the palmar surface greater than any other contiguous surface, the palmar surface and the contiguous surfaces defined by ridges.

19. A transducer housing for ultrasound imaging, the transducer housing comprising:
an acoustic window for positioning an array of transducing elements adjacent to skin of a patient, an orthogonal vector extending from a center of the acoustic window;
a palmar surface intersecting the orthogonal vector; and
a cable extending from the housing at a location spaced away from the orthogonal vector.

20. The transducer housing of claim 19 wherein the cable extends from the housing at an angle substantially perpendicular to the orthogonal vector.

21. The transducer housing of claim 19 wherein the location is on the palmar surface and the cable or a strain relief of the cable have an elongated cross-section.

22. The transducer housing of claim 19 wherein the location is between the palmar surface and the acoustic window, the palmar surface being an upper surface and the acoustic window being on an opposing lower surface.

23. The transducer housing of claim 19 further comprising:
a cable connector connecting the cable to the transducer housing, the cable connector being rotatable such that the cable may rotate relative to the transducer housing.
